(19) United States
(12) Patent Application Publication
(10) Pub. No.: US 2024/0016447 A1
(43) Pub. Date: Jan. 18, 2024

(54) SYSTEMS AND METHODS FOR GENERATING IMAGE DATA ASSOCIATED WITH A SLEEP-RELATED EVENT

(71) Applicants: ResMed Inc., San Diego, CA (US); ResMed Asia Pte. Ltd., Singapore (SG); ResMed Pty Ltd, Bella Vista, NSW (AU)

(72) Inventors: Gregory Robert Peake, Sydney (AU); Christopher Andrew Wakeley Gill, Singapore (SG); Sarah Terese McGann, Sydney (AU)

(21) Appl. No.: 18/254,659

(22) PCT Filed: Nov. 26, 2021
(86) PCT No.: PCT/SG2021/050729

§ 371 Date: May 26, 2023

Related U.S. Application Data
(60) Provisional application No. 63/118,813, filed on Nov. 27, 2020.

(51) Int. Cl. A61B 5/00

(52) U.S. Cl. CPC A61B 5/4818; A61B 5/7282; A61B 5/0077; A61B 2562/0204

(57) ABSTRACT

A method includes receiving physiological data associated with a sleep session of a user. The method also includes identifying a triggering event based at least in part on the physiological data. The method also includes generating image data in response to identifying the triggering event, the image data being reproducible as one or more images of at least a portion of the user during the sleep session. The method also includes causing at least a portion of the image data to be communicated to the user subsequent to the sleep session.

### CROSS-REFERENCE TO RELATED APPLICATIONS

This application claims the benefit of, and priority to, U.S. Provisional Patent Application No. 63/118,813, filed on Nov. 27, 2020, which is hereby incorporated by reference herein in its entirety.

### TECHNICAL FIELD

The present disclosure relates generally to systems and methods for identifying an event experienced by a user during a sleep session, and more particularly, to systems and methods for generating image data in response to identifying a triggering event.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, insomnia, and chest wall disorders. These disorders are often treated using a respiratory therapy system. However, some users find such systems to be uncomfortable, difficult to use, expensive, aesthetically unappealing and/or fail to perceive the benefits associated with using the system. As a result, some users will elect not to begin using the respiratory therapy system or discontinue use of the respiratory therapy system absent a demonstration of the severity of their symptoms when respiratory therapy treatment is not used. The present disclosure is directed to solving these and other problems.

### SUMMARY

According to some implementations of the present disclosure, a method includes receiving physiological data associated with a sleep session of a user. The method also includes identifying a triggering event based at least in part on the physiological data. The method also includes generating image data in response to identifying the triggering event, the image data being reproducible as one or more images of at least a portion of the user during the sleep session. The method also includes causing at least a portion of the image data to be communicated to the user subsequent to the sleep session.

According to some implementations of the present disclosure, a system includes an electronic interface, a memory, and a control system. The electronic interface is configured to receive data associated with a sleep session of a user. The memory stores machine-readable instructions. The control system includes one or more processors configured to execute the machine-readable instructions to determine that the user is experiencing an event based at least in part on the data associated with the sleep session of the user. The control system is further configured to cause a camera to generate image data in response to determining that the user is experiencing the event, the image data being reproducible as one or more images of at least a portion of the user during the sleep session. The control system is further configured to cause at least a portion of the image data to be communicated to the user subsequent to the sleep session.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 is a perspective view of a mount and user device of the system of FIG. 1, according to some implementations of the present disclosure;

FIG. 4 illustrates an exemplary timeline for a sleep session, according to some implementations of the present disclosure;

FIG. 5 illustrates an exemplary hypnogram associated with the sleep session of FIG. 3, according to some implementations of the present disclosure;

FIG. 6 is a process flow diagram for a method of generating image data during a sleep session in response to a triggering event, according to some implementations of the present disclosure;

FIG. 7 illustrates an exemplary respiration signal and an exemplary audio signal associated with a user during a sleep session, according to some implementations of the present disclosure; and

FIG. 8 is a flowchart depicting a process for generating event movement data during a sleep session in response to a triggering event, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hyper tension, diabetes, stroke, insomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

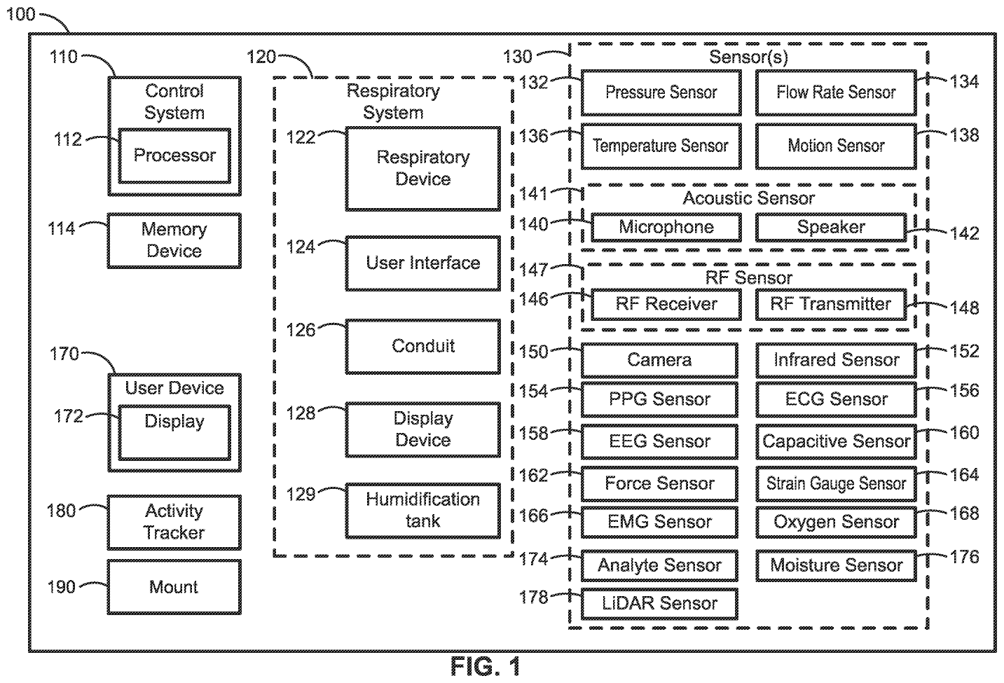

Referring to FIG. 1, a system 100, according to some implementations of the present disclosure, is illustrated. The system 100 includes a control system 110, a memory device 114, one or more sensors 130, and one or more user devices 170. In some implementations, the system 100 further optionally includes a respiratory therapy system 120, an activity tracker 180, and a mount 190.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 112 of the control system 110 and/or memory device 114 can receive data (e.g., physiological data and/or audio data) from the one or more sensors 130 such that the data for storage in the memory device 114 and/or for analysis by the processor 112. The processor 112 and/or memory device 114 can communicate with the one or more sensors 130 using a wired connection or a wireless connection (e.g., using an RF communication protocol, a Wi-Fi communication protocol, a Bluetooth communication protocol, over a cellular network, etc.). In some implementations, the system 100 can include an antenna, a receiver (e.g., an RF receiver), a transmitter (e.g., an RF transmitter), a transceiver, or any combination thereof. Such components can be coupled to or integrated a housing of the control system 110 (e.g., in the same housing as the processor 112 and/or memory device 114), or the user device 170.

As noted above, in some implementations, the system 100 optionally includes a respiratory therapy system 120 (also referred to as a respiratory therapy system). The respiratory therapy system 120 can include a respiratory pressure therapy device 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

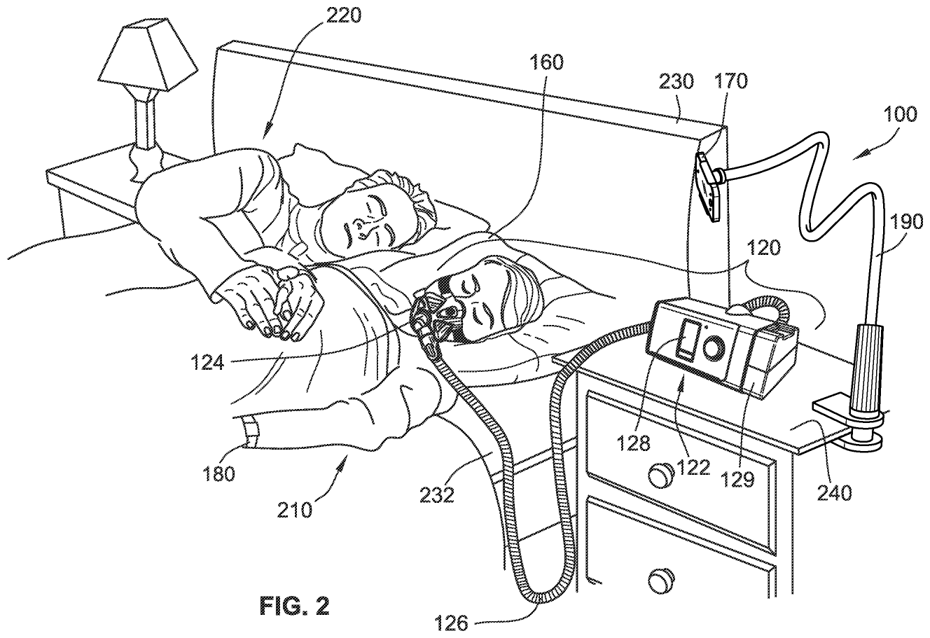

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user.

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. The conduit 126 includes a first end coupled to an outlet of the respiratory therapy device 122 and a second opposing end coupled to the user interface 124. The conduit 126 can be coupled to the respiratory therapy device 122 and/or the user interface 124 using a variety of techniques (e.g., a press fit connection, a snap fit connection, a threaded connection, etc.). In some implementations, the conduit 126 includes one or more heating elements that heat the pressurized air flowing through the conduit 126 (e.g., heat the air to a predetermined temperature or within a range of predetermined temperatures). Such heating elements can be coupled to and/or imbedded in the conduit 126. In such implementations, the end of the conduit 126 coupled to the respiratory therapy device 122 can include an electrical contact that is electrically coupled to the respiratory therapy device 122 to power the one or more heating elements of the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidifier 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidifier 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof. The sleep-wake signal can be measured by the sensor(s) 130 during the sleep session at a predetermined sampling rate, such as, for example, one sample per second, one sample per 30 seconds, one sample per minute, etc. Methods for determining sleep states and/or sleep stages from physiological data generated by one or more sensors, such as the one or more sensors 130, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory therapy device 122, the use interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, the mount 190 or any combination thereof. For example, the microphone 140 and speaker 142 is integrated in and/or coupled to the user device 170 and the pressure sensor 130 and/or flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display 172 is generally used to display image(s) including still images, video images, or both. In some implementations, the display 172 acts as a human-machine interface (HMI) that includes a graphic user interface (GUI) configured to display the image(s) and an input interface. The display 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory therapy system 120, and/or the user device 170.

The mount 190 is generally used to position the camera 150 such that at least a portion of a user of the system 100 is generally positioned within a field of view of the camera 150. In this manner, the camera 150 can generate image data reproducible as one or more images (e.g., still images, video images, or both) of the portion of the user within the field of view (e.g., during a sleep session, as described in further detail herein). For example, the camera 150 can be coupled to the mount 190 such that at least a portion (e.g., the face) of the user 210 (FIG. 2) is within the field of view of the camera 150 when the user 210 is in the bed 230.

In some implementations, the camera 150 is directly coupled to the mount 190. In other implementations, the camera 150 is indirectly coupled to the mount 190. For example, the camera 150 can be coupled to or integrated in the user device 170, and the user device 170 is directly coupled to the mount 190.

In some implementations, the mount 190 can include one or more adjustable portions that are movement relative to other portions of the mount 190 to orient the field of view of the camera 150 with respect to a target (e.g., a face of a user). For example, the one or more adjustable portions can be manually adjusted by the user to orient the field of view of the camera 150.

Alternatively, in some implementations, the one of more adjustable portions can be automatically adjusted to orient the field of view of the camera 150 with respect to a target. For example, the mount 190 can include one or more actuators (e.g., motors, servo motors, microelectromechanical systems (MEMS) devices, etc.) that are controlled by the control system 110. In such implementations, image data generated by the camera 150 can be analyzed by the control system 110 to determine whether the target is within the field of view of the camera 140 (e.g., using a facial recognition algorithm, an object recognition algorithm, etc.). If the target is not within the field of view of the camera 150, the control system 110 can cause movement of the one or more adjustable portions of the mount 190 to modify the orientation and/or position of the camera 150 until it is determined that the target is within the field of view.

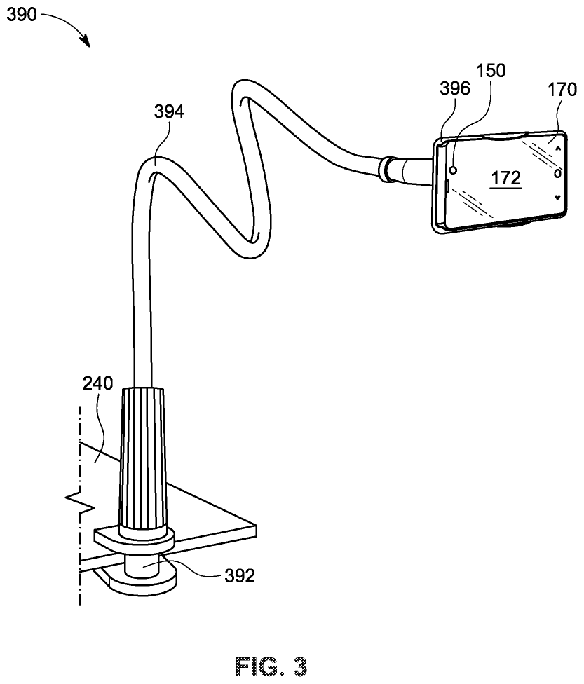

Referring to FIG. 3, a mount 390 according to one implementation of the present disclosure is illustrated. The mount 390 includes a clamp 392, an adjustable stem 394, and a bracket 396. The clamp 392 is generally used to couple the mount 390 to a surface. For example, the clamp 392 can be used to couple the mount 390 to the nightstand 240 (FIG. 2) or another other surface adjacent to the user 210 during a sleep session (e.g., the bed 230, a wall, a ceiling, other furniture, etc.). The adjustable stem 394 couples the clamp 392 and the bracket 396 and is movement relative to the clamp 392, which remains stationary. The adjustable stem 394 can be moved (e.g., by a user) to orient and/or position the bracket 396 relative to the clamp 392 as desired. The user device 170 described herein (in which the camera 150 can be integrated) can be coupled to the bracket 396. In some implementations, the bracket 396 is adjustable such that the bracket 396 is configured to receive and couple to user devices of varying shapes and sizes.

As described herein, the user device 170 can be coupled to the mount 390 during a sleep session such that the camera 150 can generate image data of at least a portion of the user (e.g., the face of the user) during the sleep session. However, users typically charge the user device 170 (e.g., smartphone) while sleeping. Thus, in some implementations, the mount 390 can include a charging cable. For example, the charging cable can be integrated in or disposed within the adjustable stem 394. In other implementations, the mount 390 can include a wireless charger (e.g., induction charger) that is coupled to or integrated in the bracket 396 to charge the user device 170 when coupled to the mount 390.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, the user device 170, and the mount 190. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display 172 of the user device 170 (FIG. 1) to manually initiate or terminate the sleep session.

Generally, the sleep session can include any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake.

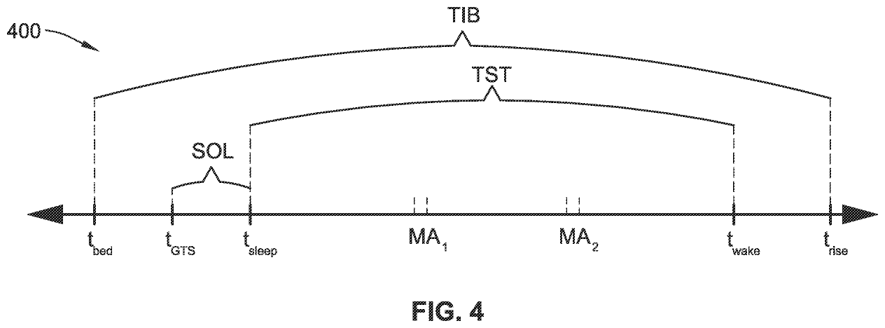

Referring to FIG. 4, an exemplary timeline 400 for a sleep session is illustrated. The timeline 400 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

The enter bed time bed is associated with the time that the user initially enters the bed (e.g., bed 230 in FIG. 2) prior to falling asleep (e.g., when the user lies down or sits in the bed). The enter bed time teed can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (bed). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least minutes, at least 20 minutes, at least 30 minutes, at least 1 hour, etc.). The enter bed time bed time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

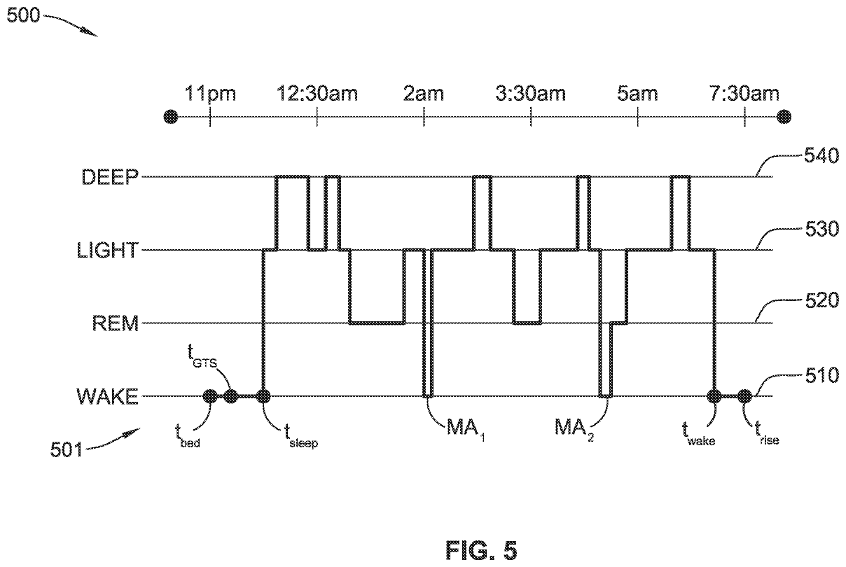

Referring to FIG. 5, an exemplary hypnogram 500 corresponding to the timeline 400 (FIG. 4), according to some implementations, is illustrated. As shown, the hypnogram 500 includes a sleep-wake signal 501, a wakefulness stage axis 510, a REM stage axis 520, a light sleep stage axis 530, and a deep sleep stage axis 540. The intersection between the sleep-wake signal 501 and one of the axes 510-540 is indicative of the sleep stage at any given time during the sleep session.

The sleep-wake signal 501 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 500 is shown in FIG. 5 as including the light sleep stage axis 530 and the deep sleep stage axis 540, in some implementations, the hypnogram 500 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 500 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 5), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 5), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time teed can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

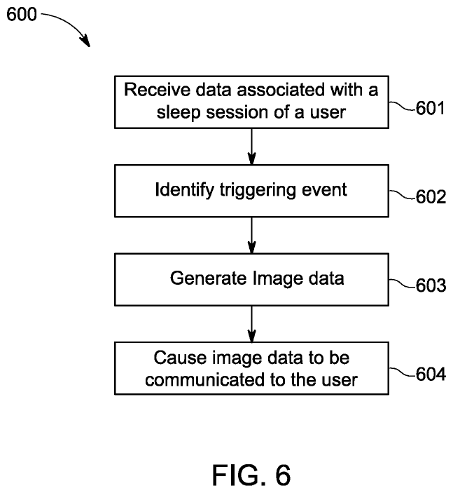

Referring to FIG. 6, a method 600 for generating image data associated with a sleep-related event according to some implementations of the present disclosure is illustrated. One or more steps of the method 600 can be implemented using any element or aspect of the system 100 (FIGS. 1-2) described herein.

Step 601 of the method 600 includes generating and/or receiving data associated with a sleep session of a user. The data can be received from the one or more sensors 130 by, for example, control system 110, the user device 170, and/or the activity tracker 180 (FIG. 1) described herein. The data can include, for example, physiological data associated with the user. The physiological data can be indicative of, among other things, respiration of the user (e.g., a respiration rate, a respiration rate variability, a tidal volume, an inspiration amplitude, an expiration amplitude, and/or an inspiration-expiration ratio) during at least a portion of the sleep session (e.g., at least 10% of the sleep session, at least 50% of the sleep session, 75% of the sleep session, at least 90% of the sleep session, etc.). The physiological data can be generated by and/or obtained from one or more of the senor(s) 130 (FIG. 1) described herein. The data can also include audio data that is reproducible as one or more sounds recorded during the sleep session (e.g., snoring, choking, breathing, a pause in breathing, labored breathing, etc.). In some implementations, the physiological data is generated by a first one of the one or more sensors 130 and the audio data is generated by a second one of the one or more sensors 130. For example, the respiration data can be generated by the pressure sensor 130 and/or flow rate sensor 132 and the audio data can be generated by the microphone 140. In this example, the pressure sensor 130 and/or the flow rate sensor 132 can be coupled to or integrated in any component or aspect of the respiratory therapy system 120 described herein, while the microphone 140 can be coupled to or integrated in the user device 170.

Step 602 of the method 600 includes identifying a triggering event based at least in part on the data received during step 601. For example, the control system 110 can analyze the received data (step 601) to identify the triggering event. As described in further detail below, the identification of the triggering event can be used initiate generation of image data. In some implementations, the triggering event can be identified using a machine learning algorithm. In such implementations, the machine learning algorithm can be trained (e.g., using supervised or unsupervised learning) to receive the data (step 601) and output an identification of one or more events. For example, the event can be identified based on data from a pulse oximeter sensor.

In some implementations, identifying the triggering event includes determining that the user is currently experiencing an event during the sleep session. In other implementations, identifying the triggering event includes predicting the user is about to experience an event (e.g., the user is about to experience an event in 1 second, 5 seconds, 10 seconds, 30 seconds, 1 minute, 3 minutes, etc.) during the sleep session. The event can include snoring, an apnea, a central apnea, an obstructive apnea, a mixed apnea, a hypopnea, a restless leg, a sleeping disorder, choking, labored breathing, an asthma attack, an epileptic episode, a seizure, or any combination thereof.

In some implementations, the method 600 further includes (e.g., as part of step 602) determining a respiration signal associated with the user during the sleep session based at least in part on the data received during step 601. For example, the control system 110 can analyze the data (e.g., that is stored in the memory device 114) received during step 601 to determine the respiration signal associated with the user during the sleep session. Information associated with and/or describing the determined respiration signal can be stored in the memory device 114 (FIG. 1), for example. The determined respiration signal can be used, for example, to aid in identifying the triggering event (e.g., whether the user is experiencing or is about to experience an event).

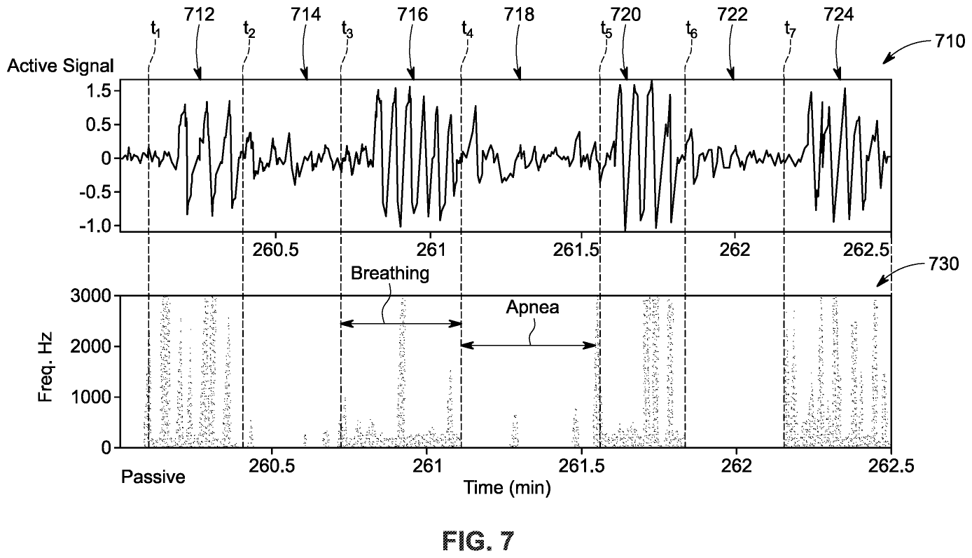

Referring to FIG. 7, an exemplary respiration signal 710 associated with a user during a portion of a sleep session is illustrated. The y-axis represents the amplitude of the respiration signal 710 and the x-axis represents time (e.g., in minutes). The respiration signal 710 includes a plurality of inhalation portions and a plurality of expiration portions. Each inhalation portion corresponds to the user breathing in (inspiration) and each exhalation portion corresponds to the user breathing out (expiration). In some implementations, the integral of one of the inhalation portions of the respiration signal 710 is equal to the integral of a corresponding one of the exhalation portions of the respiration signal 710. In the example shown in FIG. 7, the respiration signal 710 includes a first portion 712 between time t₁ and time t₂, a second portion 714 between time t₂ and time t₃, a third portion 716 between time t₃ and time t₄, a fourth portion 718 between time t₄ and time t₅, a fifth portion 720 between time t₅ and time t₆, a sixth portion 722 between time t₆ and time t₇, and a seventh portion 724 between time t₇ and time t₅.

The respiration signal 710 can be indicative of, among other things, one or more events experienced by the user during the portion of the first sleep session. For example, the first portion 712, the third portion 716, the fifth portion 720, and the seventh portion 724 of the respiration signal 710 are associated with normal breathing (e.g., one or more inhalations and one or more exhalations). The second portion 714 is associated with a first event, the fourth portion 718 is associated with a second event, and the sixth portion 720 is associated with a third event. In this particular example, the first event, second event, and third event are obstructive sleep apnea (OSA) events. These events can be identified within the respiration signal 710, for example, based on the relative amplitude of the respiration signal 710 in the first portion 712, the third portion 716, the fifth portion 720, and the seventh portion 724 relative to the second portion 714, the fourth portion 718, and the sixth portion 720. As another example, each event in the respiration signal 710 can be identified by comparing the amplitude of the respiration signal 710 to a predetermined threshold. For example, an average of the amplitude of the respiration signal 710 for a predetermined time period or sampling rate (e.g., 1 second, 3 seconds, 5 seconds, 10 seconds, 30 seconds, 1 minute, 3 minutes, etc.) can be compared to the predetermined threshold to identify one or more events during the sleep session.

In some implementations, step 602 includes identifying a single event during a portion of the sleep session, and this event is the triggering event. In other implementations, step 503 includes identifying a plurality of events during all or a portion of the sleep session, and one of these events is the triggering event. For example, referring to the exemplary respiration signal 710 of FIG. 7, three separate events were identified: a first event occurring in the second portion 714, a second event occurring in the fourth portion 718, and a third event occurring in the sixth portion 722. Accordingly, in some implementations, the triggering event is identified when an apnea-hypopnea index (AHI) is equal to or greater than a predetermined threshold value. As described above, the AHI is calculated by dividing the number of apnea and/or hypopnea events experienced by the user during the sleep session by the total number of hours of sleep in the sleep session. In such implementations, the triggering event is identified if the AHI is equal to or greater than the predetermined threshold value. The predetermined threshold value can be, for example, an AHI of about 15. In another example, the trigger event can be identified if the user stops breathing (e.g., as indicated by silence in the audio data) for a predetermined duration (e.g., more than 5 seconds, more than 10 seconds, more than 15 seconds, etc.). In some implementations, identifying the triggering event includes using a linear regression algorithm. The linear regression algorithm can be used, for example, to determine a percentage likelihood for each identified event being an actual obstructive sleep apnea (OSA) event.

In some implementations, the method 600 (e.g., as part of step 602) also includes determining an audio signal associated with the sleep session based at least in part on audio data received during step 601. Referring to FIG. 7, an exemplary audio signal 730 associated with the user during a portion of a sleep session is illustrated. The y-axis represents the frequency of the audio signal 730 and the x-axis represents time (e.g., in minutes). As illustrated in FIG. 7, the audio signal 730 generally corresponds to the respiration signal 710. In particular, the frequency of the audio signal 730 generally corresponds with (e.g., is correlated with) the amplitude of the respiration signal 710. For example, the average amplitude of the first portion 712 of the respiration signal 710 between time t₁ and t₂ is greater than the average amplitude of the second portion 614 of the audio signal 710 between time t₂ and t₃ (e.g., which is indicative of a pause in breathing between time t₂ and t₃). Likewise, the average frequency of the audio signal 730 between time t₁ and t₂ is greater than the average frequency of the audio signal 730 between time t₂ and t₃ (e.g., which is indicative of a pause in breathing between time t₂ and t₃).

The determined audio signal can be used to aid in identifying the triggering event (e.g., that the user is experiencing an event or is about to experience an event). In such implementations, the audio data can be analyzed (e.g., by the control system 110) to detect or measure respiration or breathing of the user. The audio data can also be analyzed to detect or identify a reduction or decrease in the frequency and/or amplitude of the audio, which can be indicative of a temporary cessation or pause in breathing due to an obstructive sleep apnea (OSA) event, for example. Thus, the event can be identified responsive to determining that the frequency and/or amplitude of the audio data falls below a predetermined threshold for a predetermination duration (e.g., between about 10 seconds and about 45 seconds, between about 15 seconds and about 30 seconds, etc.).

A reduction in the audio amplitude, however, can be attributable to a change in the ambient noise level in the room in which the user is sleeping rather than the user experiencing an event (e.g., a pause in breathing). Environmental factors that can affect the ambient noise include, for example, ventilation or air flow (e.g., from an HVAC system, a fan, a humidifier, a dehumidifier, etc.), household appliances (e.g., television, speakers, etc.), noise outside of the room (e.g., roommates, neighbors, traffic, etc.), or the like. As such, analyzing the audio data can include filtering ambient noise(s) from the audio data to identify the respiration and/or breathing of the user. Ambient noise can be filtered, for example, using a machine learning algorithm.

Further, in some implementations, step 602 can also include determining a position and/or orientation of the user relative to the one of the one or more sensors 130 that is generating the data. As an example, the position and/or orientation of the user can be used in aid in the generation of image (as described further below). As another example, the user may turn or move away from the sensor generating the audio data during the sleep session, which may cause a corresponding reduction of the audio amplitude. However, even as the amplitude of the audio signal may be reduced based on the position of the user relative to the sensor, the relative changes in the amplitude of the audio signal in response to an event (e.g., OSA event) are generally the same. Thus, the determined position and/or orientation of the user can be used to modify any of the predetermined audio thresholds described herein.

In some implementations, the method 600 also includes (e.g., as part of step 602) determining one or more sleep-related parameters associated with the user during the sleep session based at least in part on the received data (step 601). The one or more sleep-related parameters, for example, an apnea-hypopnea index (AHI), a number of events per hour, a pattern of events, a total sleep time, a total time in bed, a wake-up time, a rising time, a hypnogram, a total light sleep time, a total deep sleep time, a total REM sleep time, a number of awakenings, a sleep-onset latency, or any combination thereof. In some implementations, the one or more sleep-related parameters can include a sleep score, such as the ones described in International Publication No. WO 2015/006364, which is hereby incorporated by reference herein in its entirety. The one or more sleep-related parameters can include any number of sleep-related parameters (e.g., 1 sleep-related parameter, 2 sleep-related parameters, 5 sleep-related parameters, 50 sleep-related parameters, etc.).

Step 603 of the method 600 (FIG. 6) includes causing image data to be generated in response to identifying the triggering event (step 602). The image data is reproducible as one or more images (e.g., video images) of the user during the sleep session that capture at least a portion of the triggering event. The image data can be generated by the camera 150 and stored in the memory 114.

In some implementations, the camera 150 is not generating image data prior to the identification of the triggering event. Upon identifying the triggering event, the control system 110 can actuate the camera 150 to cause the camera 150 to generate image data. In other implementations, the camera 150 is generating image data prior to the identification of the triggering event. In such implementations, the generated image data may not be persistently stored before being deleted (e.g., at or before the end of the sleep session) to save space in the memory 114. However, upon identification of the triggering event, in such implementations, the generated image data can be persistently stored in the memory 114 such that the image data can be communicated to the user (step 604) and/or otherwise accessed after the sleep session.

In some implementations, the method 600 further includes causing the camera 150 to be positioned and/or orientated relative to the user such that at least a portion of the user (e.g., the face) is within the field of view. For example, the method 600 can include causing a prompt to be communicated to the user (e.g., via the user device 170) to manually move the camera 150 to a predetermined position and/or orientation, move the mount 190 to a predetermined position and/or orientation, or both. In this example, the camera 150 can generate image data before the sleep session, and the control system 110 can analyze the generated image data to determine whether the user is within the field of view (e.g., using a facial recognition algorithm) and continue to prompt the user to move the camera 150 and/or mount 190 until the control system 110 determines the user is within the field of view of the camera 150. In another example, the method 600 can include automatically causing the camera 150 to be positioned and/or orientated such that the user is within the field of view (e.g., using the mount 190, as described above).

In some implementations, the method 600 further includes determining that the user is no longer experiencing the event based at least in part on the data in the same or similar manner as identifying the triggering event. In such implementations, in response to determining that the user is no longer experiencing the event, the method 600 can include ceasing generating the image data. For example, the control system 110 can cause the camera 150 to stop generating image and/or cause image data to no longer be stored in the memory 114.

In some implementations, the method 600 further includes causing a light source (e.g., a lamp on the nightstand 240 or other furniture, a light source (e.g., LED) in the user device 170 or the display 172 of the user device 170, other light(s) in the room, etc.) to be actuated in response to identifying the triggering event to aid in generating the image data. Often, there is little or no ambient lighting during the sleep session as the lights are shut off, curtains are drawn, the sun is down, etc. Some users may find it difficult to fall asleep or stay asleep in early stages of sleep with ambient lighting (e.g., from a light source). Actuating the light source according to sleep stage may allow a user to stay asleep while having sufficient lighting to aid in generating the image data. Causing the light to be actuated in response to the triggering event can thus aid in generating the image data such that the user can see his or herself sleeping when viewing the image data. Further, such implementations, can include modifying a brightness or intensity of a light source in response to identifying the event. Alternatively, a night vision filter can be applied to the image data.

Step 604 of the method 600 (FIG. 6) includes causing at least a portion of the generated image data (step 603) to be communicated to the user subsequent to the sleep session. The image data can be communicated to the user via, for example, the user device 170 (e.g., the display 172 and/or the speaker 142). As described above, the image data is generated in response to the triggering event. Thus, displaying the image data allows the user to see his or herself during the sleep session experiencing the event.

Some users of the respiratory therapy systems described herein (e.g., CPAP systems) find such systems to be uncomfortable, difficult to use, expensive, and/or aesthetically unappealing. Some users of these systems also may not immediately notice any benefits of use after first beginning therapy. As a result, these users may choose not to use their respiratory therapy system as prescribed (e.g., every night), or even completely discontinue use of the respiratory therapy system altogether. Indeed, some users may altogether avoid seeking diagnosis and/or treatment for symptoms associated with a condition that may require use of a respiration therapy system.

While events like snoring, choking, or labored breathing (e.g., that can occur more frequently when the user does not use the respiratory therapy system) can be quite noticeable (e.g., from the perspective of the bed partner 220 in FIG. 2), the user does not directly perceive these event because they are asleep. The user may be more likely or encouraged to seek treatment, use a respiratory therapy system and/or adhere to recommended respiratory therapy in the future to reduce or eliminate these events if they could actually see and hear these events and understand the severity of their symptoms. For example, the time period where the user stops breathing during an OSA event can be between about 15 seconds and about 30 seconds. If the user watches a video of the period of silence where they stopped breathing, they would better understand the severity or seriousness of their symptoms and be more likely to seek treatment and/or use the respiratory therapy system as prescribed. Thus, communicating the image data to the user can aid in encouraging or eliciting a behavioral response by the user, such as using their respiratory therapy system as prescribed, seeking diagnosis and treatment for their symptoms, and/or otherwise changing their sleep habits.

In some implementations, step 604 includes causing additional indications or information associated with the triggering event (e.g., a description of the triggering event) to be communicated to the user. In such implementations, the additional indications can additionally or alternatively include information describing one or more determined sleep-related parameters for the sleep session (e.g., sleep score). Further, step 604 can include causing a graphical representation of the determined respiration signal to be communicated to the user (e.g., before, simultaneous with, or after the image data).

In some implementations, the method 600 further includes modifying the generated image data (step 603) and causing the modified image data to be communicated to the user. For example, the modifying the generated image data can include applying a night vision filter. The night vision filter can aid in allowing the user to view him or herself in poorly lit conditions when the modified image data is communicated to the user. As another example, where the image data includes video, the modifying the generated image data can include modifying a playback speed of the video during step 604 (e.g., slowing down or speeding up).

In some implementations, the method 600 further includes analyzing the generated image data (step 603) to identify a portion of the user (e.g., using a machine learning algorithm). The portion of the user can include, for example, a face of the user, a neck of the user, a throat of the user, a chest of the user, a nose of the user, one or more eyes of the user, or any combination thereof. In such implementations that include modifying the generated image data, the modifying can include zooming in on the identified portion of the user (e.g., the face of the user).

In some implementations, steps 601-604 can be repeated for one or more additional sleep sessions subsequent to the first sleep session (e.g., a third sleep session, a fourth sleep session, a tenth sleep session, a one-hundredth sleep session etc.). While the method 600 has been shown and described herein as occurring in a certain order, more generally, the steps of the method 600 can be performed in any suitable order.

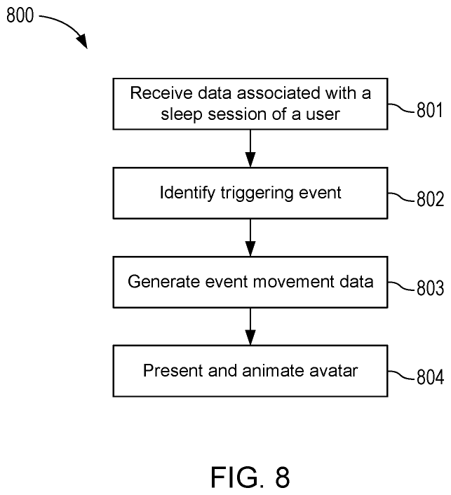

FIG. 8 is a flowchart depicting a method 800 for generating event movement data during a sleep session in response to a triggering event, according to some implementations of the present disclosure. Method 800 can be practiced using any suitable system, such as system 100 of FIG. 1. Method 800 can be similar to method 600, except where method 600 involves causing image data to be communicated to the user, method 800 involves presenting and animating an avatar representative of the user to the user.

Aspects and features of method 800 relate to the use of collected data to identify a triggering event and present and animate an avatar of the user based on that identified triggering event. The avatar can be a digitally generated simulation of the user (e.g., at least a portion of the user, such as the user's face or head), which can be rendered in any suitable fashion. For example, an avatar can be a cartoon-like figure intended to look similar to the user. As another example, an avatar can be a realistic (e.g., photorealistic) looking rendering of the user. The avatar can be controlled (e.g., animated) using movement data (e.g., event movement data) to simulate the avatar experiencing an event (e.g., a triggering event). Thus, aspects and features of method 800 can be used to present, after a sleep session, an animated version of the user choking, coughing, snoring, or otherwise engaging in events that were detected during the sleep session.

At step 801, data associated with a sleep session of a user is received. In some cases, receiving data at step 801 is the same as or similar to step 601 of FIG. 6. For example, the data can be received from one or more sensors (e.g., sensor(s) 130 of FIG. 1) and can include physiological data associated with the user.

At step 802, a triggering event can be identified. In some cases, identifying a triggering event at step 802 is the same or similar to step 602 of FIG. 6. For example, a control system (e.g., control system 110 of FIG. 1) can analyze the data received during step 801 to identify the triggering event. For example, physiological data received during step 801 can be used to determine a respiration signal (e.g., respiration signal 710 of FIG. 7), which can be used to determine a triggering event.

At step 803, event movement data can be generated. Event movement data is movement data that is associated with an event (e.g., the triggered event), such as movement data associated with an apnea, snoring, or the like. Event movement data can be based on actual movement data (e.g., based on actual measured movements of the user undergoing the event) or estimated movement data (e.g., based on a preset or generic pattern of movements). As described in further detail below, the event movement data can be leveraged to animate an avatar. For example, event movement data associated with an apnea event can be used to simulate an avatar undergoing an apnea event.

Generation of event movement data can be in response to identification of the triggering event at block 802. In some cases, generating event movement data can include using the received data from step 801 to identify movement of the user's body before, during, and/or after the triggering event.

In some cases, received data can be used to extract user movement data, which can be data that is indicative of actual movement of the user experiencing the triggering event, such as movement of the user's chest, limbs, head, mouth, and the like. This movement can be used to generate the event movement data. For example, in some cases, the event movement data can be thus generated such that an avatar animated using that event movement data will move the same way as the user moved during the triggering event.

In some cases, received data can be used to identify what type or types of triggering event(s) has occurred. For example, a triggering event may be snoring, an apnea, a central apnea, an obstructive apnea, a mixed apnea, a hypopnea, a restless leg, a sleeping disorder, choking, labored breathing, an asthma attack, an epileptic episode, a seizure, or any combination thereof. Other types triggering events as disclosed herein can be identified. In such cases, the type of triggering event can be used to select what estimated event movement data to use in generating the event movement data. For example, a number of estimated event movement data presets can be established, each for a different type or combination of types of event. In such an example, once a type is determined (e.g., apnea or snoring), the estimated event movement data preset for that type (e.g., a first preset for an apnea event and a second preset for a snoring event) can be used as the event movement data. Thus, when the system detects the user is snoring, a first estimated event movement data can be used to animate an avatar, but when the system detects the user is having an apnea event, a different, second estimated event movement data can be used to animate the avatar. In some case, merely identifying the type of event can be used to generate the event movement data. However, in some cases, once the type of event has been identified, the intensity of, duration of, or other parameter associated with the event (e.g., as determined from the received data, such as from the received physiological data) can be used to control generation of the event movement data. For example, the event movement data generated in response to a user snoring at a first volume might be different than the event movement data generated in response to the user snoring at a louder volume or for a longer duration. In such cases, even if preset estimated event movement data is used, generation of the event movement data can nonetheless be tailored to how the user experiences the event.

At step 804, an avatar can be presented and animated. Presenting the avatar can include displaying the avatar on a display device (e.g., display device 128 or display 172 of FIG. 1) or otherwise communicating the avatar and its animation to the user. The avatar can be a two or three dimensional representation of the user. In some cases, the avatar can be made to look similar to the user, although that need not always be the case. The avatar can be a full body or a portion of a body, such as only a head or only a face.

Presenting and animating the avatar can include applying the event movement data from step 803 to a model. The model can be a two or three dimensional model capable of receiving inputs to output the avatar (e.g., to output a rendering of the avatar). In some cases, a singular preset model can be used. In some cases, the user can select from a collection of preset models to use. In some cases, the user can provide user inputs to create and/or modify a model, such as to alter features of the model to appear similar to the user. In some cases, the user can provide one or more images of the user, which can be used to facilitate generation or modification of a model. For example, one or more images of a user can be mapped onto a surface of a model, permitting the model to generate an avatar that looks like the user. In some cases, two or three dimensional data of the user (e.g., of a portion of the user, such as the user's face) can be acquired, such as from one or more sensors (e.g., sensor(s) 130 of FIG. 1), and used to generate or modify a model.

In some cases, presenting the avatar can include overlaying the avatar on a background. In some cases, the background can be a preset or user-selectable background. In some cases, however, the background can be an image of the user's sleeping environment (e.g., the user's bed or bedroom, a couch if the user is sleeping on a couch, or otherwise). Such an image can be acquired during a setup procedure (e.g., the system can ask the user to capture or provide an image of the user's bed or bedroom) or during a sleep session (e.g., the system can make use of a camera to generate image data as described in further detail herein, permitting the image data to be used as the image of the sleeping environment).

The avatar can be animated based on the event movement data from step 803. The event movement data, whether based on estimated event movement data presets or based on actual user movement, can be applied to the model to animate the avatar according to the event movement data. For example, if the triggered event is an apnea event, the event movement data can be representative of the user breathing and then temporarily stopping breathing for a period of time, and when applied to a model, the event movement data can result in the avatar being animated to breath and then temporarily stop breathing for a period of time.

In some cases, the event movement data can be further modified, such as to emphasize certain events or adjust the speed of a “recorded” event. In an example, in response to identifying a triggering event, the system can capture audio data and generate event movement data for a period of time associated with the triggering event (e.g., from three seconds before identification of the triggering event until three seconds after the triggering event has been determined to cease). In such an example, a temporal compression factor can be applied to both the audio data and the event movement data such that when the avatar is present, it can be animated in time with the audio data. A temporal compression factor can be a degree to which a time-dependent data stream (e.g., audio data or event movement data) is expanded or compressed temporally. For example, thirty seconds of audio data can be played back in fifteen seconds with a 2× temporal compression factor, or in sixty seconds with a 0.5× temporal compression factor.

In some cases, animating an event can include emphasizing the event. For example, when animating an apnea event, the avatar can be further animated to glow red to emphasize the significance of the apnea event. In some cases, the event movement data can be modified to facilitate emphasizing an event. In some cases, discernable emphasis (e.g., emphasized visual features, emphasized audio features, emphasized movements) can be added to facilitate encouraging the user to improve habits or practices such that the event(s) that were emphasized can be avoided in future sleep sessions. For example, for a user of a respiratory therapy device, if the user decides not to use the device for a night, a sleep summary the following day can show an avatar associated with the sleep session in which the respiratory therapy device was not used, and the avatar can be animated to show how the user stopped breathing for a period of time due to an apnea event. In such cases, emphasis can be provided (e.g., by modifying or adding to the rendered avatar, such as adjusting the color of the avatar) to encourage the user to make use of the respiratory therapy device on future nights.

In some cases, presenting and animating the avatar at step 804 can further include adjusting how the avatar is being presented based on user input. For example, the avatar can be presented in a fashion such that a user can dynamically interact with it, such as to spin the avatar around and see it from different angles. For example, while playing back certain triggering events from a previous night's sleep, a user may be able to tap and swipe on a touchscreen display that is displaying the avatar so as to move from seeing the avatar head-on to seeing the avatar from the side. In some cases, different views can be presented automatically or in response to user input. In some cases, different views can include rendering certain layers of the avatar (e.g., certain layers of the model of the avatar) as translucent or transparent. For example, in some cases the user may be able to switch between a standard view (e.g., a view similar to looking straight at the user's face) and an anatomical view (e.g., a view where portions of the user's skin, muscle, and/or bone are rendered translucent or transparent so that it is easier to see how certain muscles, bone, or other tissue interact during an event, such as to see how the certain tissue falls during an obstructive sleep apnea event).

In some cases, method 800 can include fewer or additional steps, such as those disclosed elsewhere herein. For example, in some cases, method 800 can additionally include steps 603 and 604, thus permitting both an avatar and image data of the user to be presented after a sleep session.

One or more elements or aspects or steps, or any portion(s) thereof, from one or more of any of claims 1 to 87 below can be combined with one or more elements or aspects or steps, or any portion(s) thereof, from one or more of any of the other claims 1 to 87 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving physiological data associated with a sleep session of a user;
identifying a triggering event based at least in part on the physiological data;
generating image data in response to identifying the triggering event, the image data being reproducible as one or more images of at least a portion of the user during the sleep session; and
causing at least a portion of the image data to be communicated to the user subsequent to the sleep session.

2. The method of claim 1, wherein the identifying the triggering event includes determining that the user is experiencing an event, wherein the event includes snoring, an apnea, a central apnea, an obstructive apnea, a mixed apnea, a hypopnea, a restless leg, a sleeping disorder, choking, labored breathing, an asthma attack, an epileptic episode, a seizure, or any combination thereof.

3. The method of claim 1, wherein the identifying the triggering event includes predicting the user is about to experience an event, wherein the event includes snoring, an apnea, a central apnea, an obstructive apnea, a mixed apnea, a hypopnea, a restless leg, a sleeping disorder, choking, labored breathing, an asthma attack, an epileptic episode, a seizure, or any combination thereof.

4. The method of claim 1, further comprising receiving audio data reproducible as one or more sounds associated with the user during at least a portion of the sleep session.

5. The method of claim 4, wherein the identifying the triggering event is based at least in part on the audio data.

6. The method of claim 5, wherein a first portion of the audio data is received prior to the identifying the triggering event and a second portion of the audio data is received in response to identifying the triggering event.

7-8. (canceled)

9. The method of claim 4, further comprising modifying the audio data and communicating the modified audio data to the user subsequent to the sleep session, wherein the modifying the audio data includes adding one or more sound effects, modifying a playback speed of the audio data, applying one or more filters, or any combination thereof.

10. (canceled)

11. The method of claim 1, wherein the image data is generated using a camera.

12. The method of claim 11, further comprising prompting the user to position the camera at a predetermined location prior to the sleep session such that the user is within a field of view of the camera during at least a portion of the sleep session.

13. The method of claim 11, further comprising prompting the user to modify an orientation of the camera prior to the sleep session such that the user is within a field of view of the camera during at least a portion of the sleep session.

14. The method of claim 11, further comprising modifying a position or orientation of the camera during the sleep session such that the user is within a field of view of the camera during at least a portion of the sleep session.

15-17. (canceled)

18. The method of claim 11, further comprising determining a position of the user during the sleep session and modifying a position or orientation of the camera relative to the user to aid in causing the user to be positioned within a field of view of the camera.

19-21. (canceled)

22. The method of claim 1, further comprising modifying at least a portion of the image data and causing the modified image data to be communicated to the user.

23. (canceled)

24. The method of claim 22, further comprising analyzing the image data to identify a portion of the user, the portion of the user including a face of the user, a neck of the user, a throat of the user, a chest of the user, a nose of the user, one or more eyes of the user, or any combination thereof.

25. (canceled)

26. The method of claim 22, wherein the image data includes video images and the modifying at the least portion of the image data includes modifying a playback speed of the video images.

27. The method of claim 1, further comprising modifying an ambient lighting to aid in generating the image data.

28-29. (canceled)

30. The method of claim 1, further comprising determining that the user is no longer experiencing the triggering event based at least in part on the physiological data and ceasing generating the image data in response to determining that the user is no longer experiencing the event.

31-34. (canceled)

35. A system comprising:
a memory storing machine-readable instructions; and
a control system including one or more processors configured to execute the machine-readable instructions to:
determine that the user is experiencing an event based at least in part on data associated with the sleep session of the user;
cause a camera to generate image data in response to determining that the user is experiencing the event, the image data being reproducible as one or more images of at least a portion of the user during the sleep session; and
cause at least a portion of the image data to be communicated to the user subsequent to the sleep session.

36. (canceled)

37. The system of claim 35, further comprising a mount configured to be coupled to the camera to aid in positioning or orientating a field of view of the camera such that at least a portion of the user is within the field of view of the camera during the sleep session.

38. (canceled)

39. The system of claim 35, further comprising a light source configured to emit light, wherein the control system is further configured to actuate the light source to cause the light source to emit light in response to determining that the user is experiencing the event to aid in generating the image data.

40-87. (canceled)
